(19) United States
(12) Patent Application Publication
(10) Pub. No.: US 2002/0165197 A1
(43) Pub. Date: Nov. 7, 2002

(54) Modulation of GSK-3beta activity and its different uses

(76) Inventors: Pnina Fishman, Herzliya (IL); Kamel Khalili, Merion, PA (US)

Correspondence Address: BIRCH STEWART KOLASCH & BIRCH, PO BOX 747, FALLS CHURCH, VA, 22040-0747, US

(21) Appl. No.: 10/078,712

(22) Filed: Feb. 21, 2002

Related U.S. Application Data
(63) Continuation-in-part of application No. 09/788,477, filed on Feb. 21, 2001.

(51) Int. Cl. A61K 31/7076; A61K 31/522

(52) U.S. Cl. 514/46; 514/263.34

(57) ABSTRACT

The present invention concerns the modulation of glycogene synthase kinase 3β (G3SK-3β) by an adenosine receptor ligand. Depending on the type of ligand, the modulation may be manifested by down-regulation or up-regulation of the kinase's activity.

Thus, there is provided by the present invention a method and pharmacetical compositions for achieving a therapeutic effect involved in modulating GSK-3β activity in cells by the use of an adenosine receptor ligand or a combination of ARLs.

When modulation involves activation of GSK-3β activity the ARL may be an adenosine A1 receptor agonist (A1RAg), an adenosine A3 receptor agonist (A3RAg), an adenosine A2 receptor antagonist (A2RAn) or any combination of the same, while when modulation involves inhabition of GSK-3β activity the ARL may be an adenosine A1 receptor antagonist (A1RAn), an adenosine A3 receptor antagonist (A3RAn), an adenosine A2 receptor agonist (A2RAg) or any combination of the same.

### FIELD OF THE INVENTION

The present invention relates to therapeutic use of adenosire agonists and antagonists as GSK-3β modulators.

### LIST OF PRIOR ART

The following is a list of prior art which is considered to be pertinent for describing the state of the art in the field of the invention, all of which are identified in the description by the following reference number.

1. Linden J. The FASEB J 5:2668-2676(1991);

2. Stiles G. L. Clin, Res. 38:10-18 (1990);

3. Filippa N, et al. Mol Cell Biol. 19:4989-5000 (1999);

4. Sable CL, et al. FEBS Lett 409:253-257 (1997);

5. Fang X, et al. Proc Nat Acad Sci U S A. 97:11960-11965 (2000);

6. Cross DA, et al., Natre 378:785-789. (1995);

7. Fishman, P. et al. Eur. J. Cancer 36:1452-1458 (2000);

8. Moule SK,. et al. J Biol chem Mar 272:7713-7729 (1997);

### BACKGROUND OF TUE INVENTION

Adenosine Receptor Cascade

Adenosine is a ubiquitous nucleoside present in all body cells. It is released from metabolically active or stressed cells and subsequently acts as a regulatory molecule. It binds to cells through specific A1, A2A, A2B and A3 G-protein associated cell surface receptors, thus acting as a signal transduction imolecule by reguating the levels of adenylyl cyclase and phospholipase C^{(1,2)}. The adenosine receptors will be referred to herein after as “A1 receptor” or “A1R”, etc., The binding of adenosine and its agonists to the A3 receptor (A3R) is known to activate the Gi protein cascade, which inhibits adenylate cyclase activity and the production of cAMP. cAMP modulates the level and activity of protein kinase A and B (PKA and PKBE/Akt, respectively), which play a ceatral role in the kinase cascade induced by a variety of extracellular signals^{(3,4)}. PKAc contains a catalytic subunit, PKAc, which dissociates from the parent molecule upon activation with cAMP. Recently, is Fang et al.⁽⁵⁾ demonstrated that PKAc phosphorylates and inactivates glycogen synthase kinase-3β (GSK-3β a serne/threonine kinase that regulates glycogen synthesis in response to insulin. GSK-3β0 also serves as a direct substrate of PKB/Akt that induces its phosphorylation and inactivation⁽⁶⁾.

The Wnt Signal Transduction Pathway

The Wnt signalling pathway with its most celebrated participants, β-catenin and Lef/Tcf, has emerged as an important player in a number of neoplasia including malignant melanoma. Wnt's are a family of paracrine and autocrne factors that regulate cell growth and cell fate [Peifer M. and Polakis P. Science 287:1606-1609 (2000)]. Sigaling by the Wnt pathway is initiated when Wnt ligands bind to transnembrane receptors of the Frized family, Frizzleds (Frz) signal, through Dishevelled (Dsh) to inhibit the kinase activity of a complex containing glycogen synthase kinase 3 (GSK-3 β), APC, AXIN and other proteins. The complex targets β-catenin and phosphorylates the threonine and serine residues of exon 3. The phosphorylated β-catenin is rapidly degraded by the ubiquitin-proteasome pathway. A mutation in the serine and threonine residues of exon 3 of β-catenin prevents phosphorylation of catenin and results in stabilization of the protein. Once hypophosphorylated due to Wnt signaling occurs, stabilized β-catenin accumulates in the cells, trislocates to the nucleus where it binds to Lef/Tcf family of transcription factors, upregulates the evression of Wnt target genes including cyclin D and c-myc [Sakanaka C, et al. Recent Prog Hormi Res. 55:225-236 (2000)].

Wnt Signaling and Human Diseases

Diseases Involved With GSK-3β Deficiency

The involvement of the Wnt pathway in the development of melanoma was first discovered by the presence of a single mutation in the N-terminus of β-catenin [Robbins PF, et al. J Exp Med. 183:1185-1192 (1996)]. This discovery was supported by later reports suggesting that downstream components of the Wnt pathway such as APC (adenomatoza polyposis coli) and β-catenin, are involved in human cancer. There are also several reports that Wnt ligands are highly expressed in tumors.

In addition there are also reports that defects in the Wnt/APC/β-catenin/Tcf pathway are implicated in other neoplasm. For example, some mutations in APC which typically lead to a truncated protein with no regulatory activity can cause the accumulation of free β-catenin. Alternatively, a mutation in β-catenin can increase the half-life of β-catenin, the latter can then stimulate the transcription of cell cycle regulators such as myc and cyclin D. The level of β-catenin could be reduced by overexpression of APC in these cells, and/or enhancement in the activity of GSK-3β which causes phosphrlation of β-catenin and its degradtion [Robbins PF et al. J Exp Med. 183:1185-1192 (1996); Barker N et al. Adv Cancer Res. 77:1-24 (2000)].

Diseases Involved With GSK-3β Hyper Function

The kinase GSK-3β along with another kinase, cyclin dependent kinase (CDK5) were found to be responsible for some abnormal hypexphosphorylation of the microtubule binding protein taut observed in the neurodegenerative Alieimer's disease. Thus, it has now been suggested tat agents which hihibit GSK-3β may be useful for the treatment or prevention of not only Alzheimer's disease but also of other hyperphosphorylation related degenerative diseases, such as frontal lobe degeneration, argyrophilic grains disease, and subacute scleroting panencephalitis (as a late complication of viral infection in the central nerve system), and for the treatment of neurotraumatic diseases such as acute stroke, psychiatric (mood) disorders such as schizophrenia and manic depression.

In addition, it has been shown that elevated GSK-3β activity is involved in the development of insulin resistance and Ape It diabetes (non-insulin dependent diabetes mellitus). Thus, as now suggestesd, agents which hihibit GSK-3β activity may be used for the treatment or prevention of type II diabetes.

### SUMMARY OF THE INVENTION

The present invention has its object to provide agents, which that are capable of modulating the GSK-3β activity. These agents in accordance with the invention are agonists or antagonists of adenosine receptors.

The present invention is based on the surprising finding that ligands, either agonits or antagonists, of the adenosine receptor are capable of modulating the Wnt signal transduction pathway. For example, activation of the A3 adenosine receptor (A3AR) in melanoma cells decreased the cAMP levels, thereby preventing the activation of both PKA and PKB/Akt. Consequently, GSK-3β was not phosphorylated and remained in its active form, which led to the induction of cell cycle arrest and apoptosis.

GSK-3β and other components of the Wnt signaling transduction pathway were proposed as a target of drugs for treating a variety of human diseases or disorders, including those mentioned above. However, in order to directly target and affect them, the drug needs to enter the cell. In accordance with the invention these agents are targeted indirectly through the receptors that are presented on the surface of the target cells—the adenosine receptors.

Thus, the invention relates in its broadest sense to a method for a therapeutic treatment, comprising administering to a subject in need an effective amount of an active agent for achieving a therapeutic effect, the therapeutic effect comprises modulating GSK-3β activity in cells and said active agent is an adenosine receptor ligand (ARL).

The term “ligand” used herein refers to any molecule capable of binding to one or more of the adenosine receptors, thereby influencing the activity of tie corresponding receptor (fully or partially). The ligand according to the invention may be specific, e.g. an A1RL is a ligand which specifically binds to the adenosine A1 receptor. Alternatively, it may be the case that a ligand binds and modulates the activity of more than one receptor. For example, a ligand may be an adenosine A1 and A3 receptor agonists which are known to inhibit adenylate cyclase.

The ligand may be full agonist, full antagonist, partial agonist or partial antagonist of the adenosine receptor. As used herein, a compound is a “full agonist” of an adenosine receptor if it produces (or induces (e.g. when increased in concentration) the maximal possible response achievable by activation of this receptor. To this end, an agent according to the invention is a full agonist of an adenosine A1 or A3 receptor if it is able to fully inhibit adenylate cyclase activity, while an agent according to the invention should be considered a “full antagonist” of an adenosine A1 or A3 receptor if it is able to rally activate adenylate cyclase. In addition, a “partial agonist” is an agent, which, no matter how high a concentration is applied, is unable to produce maximal Ovation of the receptors. To this end, an agent according to the invention is a “partial agonist” of an adenosine A1 or A3 receptor if it is able to partially inhibit adenylate cyclase activity, while an agent is a “partial antagonist” of an adenosine A1 or A3 receptor if it is able to partially activate adenylate cyclase.

The specific ligand to be used depends on the target cell in the body, the type of adenosine receptors displayed on it and whether it is desired to hihibit or activate GSK-3β activity. Where it is desired to activate GSK-3β, an A1R or an A3R agonist may be used. However, if A2R is displayed on the target cells, an antagonist of A2R may be used as well, and in consequence of the blocking of this receptor, adenosine released by the target cell or by surrounding cells or delivered to tie target cell by the body's circulation, will than act only on the A1R or A3R present on these cells thus achieving a de-facto A1R or A3R agnostic activity. Similarly, for indications requiring an A2R agonist, an antagonist of one or both of A1R or A3R may be used, with a similar effect to achieve a de-facto A2A agonists activity.

Two main embodiments are provided by the present invention. The first embodiment, to be referred to herein as the “GSK-3β activation embodiment” involves enhancement of the GSK-3β activity in cells, which may have a therapeutic value for the treatment of diseases or disorders associated with GSK-3β deficiently or dysfunction. As indicated hereinbefore, it has been described that neoplasia is associated wit GSK-3β deficiency Thus, agents which are capable of enhancing GSK-3β activity may be of therapeutic use in the treatment or prevention of diseases or disorders associated with abnormal cell proliferation. To this end, the present invention provides agents, which enhance this kinase's activity. These agents are adenosine receptor ligands (ARL) non-limiting examples of which include adenosine A1 receptor agonists, adenosine A3 receptor agonists (A3RAg), adenosine A2 (including A2A and A2B) receptor antagonist (A2RAn) or any combination of A1RAg, A3RAg and A2RAn.

The second embodiment of the present invention, to be referred to herein as the “GSK-3β inhibition embodiment” involves reduction/suppression of the kinase activity, which, accordingly, may have a therapeutic value for the treatment of diseases or disorders associated with elevated GSK-3β activity. As indicated hereinbefore, there are several illnesses, which result from hyperphosphorylation by this kinase, such as Alzheimer's disease or diabetes type II. Thus, agents capable of suppressing GSK-3β activity may have therapeutic use in the treatment or prevention of such illnesses. To this end, the present invention provides agents, which inhibit GSK-3β activity. These biologically active agents are ARL, non-limiting examples of which include adenosine A1 receptor antagonists (A1RAn), adenosine A3 receptor antagonists (A3RAn), adenosine A2 (including A2A and A2B) receptor agonists (A2RAg) or any combination of A1RAn, A3RAn and The term “treatment” as used herein refers to the administering of a is therapeutic effective amount of the agent provided by the present invention, the amount being sufficient to achieve a therapeutic effect leading to amelioration of undesired symptoms associated with a disease such as hair loss, Alzheimer's disease, acute stroke, schizophrenia, manic depression, etc., prevention of the manifestation of such symptoms before they occur, slowing down the deteriotation of the symptoms, slowing down the progression of the disease, lessening the severity or caring the disease, improving of the survival rate or resulting in a more rapid recovery of a subject suffering from the disease, prevention of the disease form occurring or a combination of two or more of the above.

The “effective amount” for purposes herein is determined by such considerations as may be known in the art. The amount must be effective to achieve the desired therapeutic effect as described above, i.e. modulation of GSK-3β, depending, inter alia, on the type and severity of the disease to be treated and the treatment regime. The effective amount is typically determined in appropriately designed clinical trials (eg. dose range studies) and the person versed in the art will know how to properly conduct such trails in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

The present invention also provides pharmaceutical compositions for achieving a therapeutic effect in a subject in need, the therapeutic effect comprising modulating GSK-3β activity in target cells, the compositions comprising an effective amount of an active agent and one or more pharmaceutically acceptable additives, the active agent being an adenosine receptor ligand (ARL) or a combination of ARL.

The term “target cells” is used herein to denote the cells in which the level of GSK-3β is to be modulated, e.g. in order to achieve the desired therapeutic effect within the framework of said treatment. The target cells may be cells in which the GSK-3β level is abnormal, i.e. it is elevated or reduces as compared to the level of GSK-3β in cells of the same type under normal conditions (a non-diseased state). The target cells may at times also be normal, non-diseased cells in which modulation of GSK-3β will give rise to a desired therapeutic effect within framework of said treatment. In general, modulation of the GSK-3β level in the target cells gives rise to said treatment in a subject in need of such treatment.

The present invention also provides pharmaceutical compositions for both embodiments of the invention as defined above. Thus, in accordance with the first embodiment, i.e. the “GSK-3β activation embodiment”, the composition of the invention will comprise one or more agents capable of elevating GSK-3β activity in cells. Such agents include, for example, the A1RAg, A3RAg, A2RAn and any combination of the same. In accordance with the “GSK-3β inhibition embodiment”, the composition comprises one or more agents capable of suppressing GSK-3β activity in cells, the agent being an ARL or any combination of ARL. Examples of ARL which may be employed according to this embodiment include A1RAn, A3RAn, A2RAg. The composition of the invention also comprises, as will be readily appreciated by the artisan, one or more pharmaceutically acceptable carries, diluents or, excipients. The pharmaceutical composition may be formulated for oral parenteral, nasal or topical administration. The mode of administration depends on the bioavailability of the specific ligand that is used as the active ingredient and at times also on the indication.

The invention also provides use of a ligand as defined above for the preparation of a pharmaceutical composition for the treatment of a disease or disorder that can be treated by modulating activity of GSK-3β.

### BRIEF DESCRIPTION OF THE DRAWINGS

In order to understand the invention and to see how it may be cared out in practice, a preferred embodiment will now be described, by way of non-limiting example only, with reference to the accompanying drawings, in which:

FIG. 1 is a bar diagram showing the dose dependent inhibitory effect of IB-MECA, an A3R agonist, on the proliferation of B16-F10 melanoma cells. B16-F10 melanoma cells were treated with vehicle (control) or with various IB-MECA concentrations (0.001 μM-10 μM) in the presence of 1% FgS for 24 h. Cell proliferation was measured by [3H]-thymidine incorporation assay. The A3 adenosine receptor antagonist MRS-1523 (0.01 μM) neutralized The inhibitory effect of IB-MECA. Data points are mean±SEM values from four independent experiments.

FIGS. 2A-2B are Western immunoblots showing levels of PKAc, phosphorylated PKB/Akt (Serine 473 phosphorylated) and total. PKB/Akt as determined from cell protein extract after exposure of the cells to IB-MECA (control is from the same cells but without exposure to IB-MECA). FIG. 2A shows decrease in the level of PKAc after 10 min. and a total disappearance after 20 min.; FIG. 2B shows that the level of phosphorylated PKB/Akt was unchanged after 30 min. but disappeared in the treated cells after 3 hours, while levels of total PKB/Akt did not change throughout the assay. NS refers to non-stimulated cells.

FIG. 3A-3D are Western immunoblots showing levels of CSK-3β and β-Actin upon treatment of cells with vehicle (Control) or with 0.01 μM IB-MECA (or also 10 μM in FIG. 3B) for the times (FIG. 3A) or concentrations (FIG. 3B) indicated. The level of phosphorylated GSK-3β (GSK-3β-P) was also determined by treatment with IB-MECA (0.01 μM) (FIG. 3C). In addition, a reduction in the level of GSK-3β upon treatment with 8Br cAMP, which mimics activation of A2R was observed (FIG. 3D).

FIG. 4 is a Western immunoblot showing levels of PKAc and GSK-3β in B16F10 melanoma cell extracts, Cell cultures containing the vehicle (Control) B-MECA or IB-MECA and the A3R antagonist MRS1523 (0.01 μM) were established. 1523 blocked-the ability of IB-MCA to decrease/increase the levels of PKAc and GSK-3β receptively.

FIG. 5 is a Western immunoblot showing levels of β-catenin upon treatment of B16-F10 melanoma cells with IB-MECA. While levels of β-catenin can be easily detected in untreated cells (left lane) only low levels are detected in treated cells.

FIG. 6A-6B show, respectively, levels of cyclin D1 and c-myc in B16-F10 melanoma treated cells upon treatment with the vehicle (control) or with IB-MECA (0.01 μM).

FIG. 7A-7C are Western immunoblots of protein extracts from tumor tissue derived from HCT-116 colon carcinoma beaming mice, treated or untreated with the A3R agonist Cl-IB-MECA). The level of β-catenin (FIG. 7A), cyclin D1 (FIG. 7B) and c-mye (FIG. 7C) after modulation with Cl-IB-MECA was determined using anti-p-catenin, anti-cyclin D1 and anti-c-myc antibodies respectively. A prominent lane was detected in all samples of untreated mice (left lane “Contorl”), while in the treated group, a decreased level of the proteins is observed (right lane, “Cl-IB-ECA”).

FIG. 8 is a schematic illustration of the signaling pathway mediated by, for example only, A3RAg. A similar pathway applies for other adenosine ligands, mutatis motandis.

### DETAILED DESCRIPTION OF THE INVENTION

As will be shown in the following specific Examples an increased level of GSK-3β with a decreased p-caterin and Lef/Tcf levels were found following treatment of the B-16 melanoma cells with either IB-MECA (N⁶-iodobenzyl)-adenosine-5′-N-medily-uronamide ) or Cl-IB-MECA (2-chloro-N⁶-(2-iodobenryl)-adenosine-5′-N-methly-urmide)as well as a decrease in the level of cyclin D1, one of the end products of the Wnt pathway and a key elements of cell cycle progression.

Further, as will be shown in the following specific examples, activation of A3AR in melanoma cells led to the decrease in cAMP levels, which resulted in the inactivation of both PKA and PKB/Akt which play a central role in the kinase cascade induced by a variety of extracellular signals Consequently, GSK-3β was not phosphorylated and was left in its active form leading to an induction of cell cycle arrest and apoptosis.

It was thus realized by the inventors of the present invention that ligands of adenosine receptors, may be useful in modulating GSK-3β.

Thus, the present invention provides a method for a therapeutic treatment comprising administering to a subject in need an effective amount of an active agent for achieving a therapeutic effect, the therapeutic effect comprise modulating GSK-3β activity in cells and said active agent is an adenosine receptor ligand (ARL) or a combination of ARL.

In the case of the GSK-3β activation embodiment of the present invention, the adenosine receptor ligand may be selected from adenosine A1 receptor agonist (A1RAg), adenosine A3 receptor agonist (A3RAg), adenosine A2 receptor antagonist (A2RAn) or any combination of A1RAg, A3RAg and A2RAn.

Some of the agents of the present invention and their synthesis procedure may be found in detail in U.S. Pat. Nos. 5,688,774; 5,773,423, 5,573;772, 5,443,836, 6,048,865, WO 95/02604, WO 99/20284 and WO 99/06053, WO 97/27173.

According to one aspect of the GSK-3β activation embodiment, tie active agent is an A1RAg. Non-limiting examples of such agents include N⁶-cyclopentyl adenosine (CPA), 2-chloro-CPA (CCPA), N⁶-cyclohexyl adenosine (CHA), N⁶-(peyl-2R-lopyl)adenosine (R-PIA) and 8-{4-[({[(2-aminoethyl)amino]carbonyl}methyl)oxyl-phenyl}-1,3diprpylxanthine(XAC).

According to another aspect of the GSK-3β activation embodiment, the active agent is an A3RAg. Non-limiting examples of such agents include 2-(4-aminophenyl)etyl adenosine (APNEA), N⁶-(4-amino3-iodobenzyl) adenosine-5′-(N-methyluronamide) (AB-MECA) and N⁶-(2-iodobenzyl)adenosine-5′-N-methly-uronamide (IB-MECA) and 2-chloro-N⁶-(2-iodobenzl)-adeosine-5′-N-melly-uronamide (Cl-IB-MECA). Other A3RAg include, N⁶-benzyl-adenosine-5′-alkluronarmde-N¹-oxide or N⁶-benyladenosine-5′-N-dialyluron-amide-N¹-oxide.

Yet further, the active agent forming part of the GSK-3β actiatidn embodiment may be an A2RAn. A non-limiting example include 3,7-dimethyl-1-propargy-xantane (DMPX).

When referring to the GSK-3β inhibition embodiment, the ARL may be selected from adenosine A1 receptor antagonist (A1RAn), adenosine A3 receptor antagonist (A3RAn), adenosine A2 receptor agonist (A2RAg) and any combination of A1RAn, A3RAn and A2RAg.

According to one aspect of the GSK-3β inhibition embodiment, the active agent is an A1RAn. A non-limiting example of such an agent includes 1,3-dipropyl-8-cyclopentylxantine (DCPC).

According to a Her aspect of this embodiment the active agent is an A3RAn. Non-limiting examples of such agents include 5-propyl-2-ethyl-4-propyl-3-ethylsulfinylcarbonyl)-6-phenylpyridine-5arboxylate(MRS-1523) and 9-chloro-2-(2-furanyl)-5-[(henylacetyl)amino] [1,2,4,]-triazolo[1,5-c] quinazoline (MRS-1200).

Yet firer, A2RAg may be selected as an agent for use in the GSK-3β inhibition embodiment. Such an agent may be, without being limited thereto N⁶-[2-(3,5-dimethoxyphenyl)-2-(2-methylphenyl)-ethyl]adenosine (DMPA).

The active agents disclosed herein may be administered and dosed in accordance with good medical practice, taking into account the clinical condition of the individual patient the site and method of administration, scheduling of is administration, patient age, sex, body weight and other factors known to medical practitioners. Accordingly, the active agent may be administered orally, subcutaneously or parenterally including intravenous, intaarterial, intramuscular, intraperitoneally and intranasal administration as well as by infusion techniques. However, oral administration is preferable.

For achieving the desired therapeutic effect, the active agent may be administered as the low molecular weight compound or as a pharmaceutically acceptable salt thereof and can be administered alone in combination with pharmaceutically acceptable additives. Accordingly, the present invention also provides pharmaceutical compositions for achieving a therapeutic effect in a subject in need, the therapeutic effect comprising modulating GSK-30β activity in cells, the composition comprising a therapeutically effective amount of one or more active agents and a pharmaceutically acceptable additive, said active agent being an ARL.

The term “pharmaceutically acceptable additives” used herein refers to one or more substances combined with said active agent and include, without being limited thereto, diluents, excipients, carriers, solid or liquid fillers or encapsulating materials which are typically added to formulations to give them a form or consistency when it is given in a specific form, e.g. in pill form, as a simple syrup, aromatic powder, and other various elixirs. The additives may also be substances for providing the formulation with stability sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc.

Preferably, the additives are inert, non-toxic materials, which do not react with the active ingredient of the invention. Yet, the additives may be designed to enhance the binding of the active agent to its receptor. At times however, the additive may also include adjuvants, which, by definition, are substances affecting the action of the active ingredient in a predictable way.

The additives can be any of those conventionally used and are limited only by chemico-physical considerations, such as solubility and lack of reactivity with the compound (unless such reactively is desired, as with adjuvants), and by the route of administration.

It is noted that humans are treated generally longer than, experimental animals as exemplified herein, which treatment has a length proportional to the length of the disease process and active agent effectiveness. The doses may be single doses or multiple doses over a period of several days. The treatment generally has a length proportional to the length of the disease process and active agent effectiveness and the patient species being treated.

The active agent of the invention may be administered orally to the patient. Conventional methods such as administering the active agent in tablets, suspensions, solutions, emulsions, capsules, powders, syrups and the like are usable.

For oral administration, the composition of the invention may contain additives for facilitating oral delivery of the active agent. Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar colloidal silicon dioxide, croscarmellose sodiumk talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active agent in a flavor, usually sucrose and acacia or tragacanth, as well as pastilles comprising the active ingredient in an inert base, such as gelatin and glycerin, or sucrose and acacia, emulsions, gels, and the like. Such additives are gene known in the art.

Alternatively, the active agent may be administered to the patient parenterally. In this case, the composition will generally be formulated in a unit dosage injectable form (solution, suspension, emulsion). Pharmaceutical formulation suitable for injection may include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, lipid polyethylene glycol and the like), suitable mixtures thereof; a vegetable oil such as cottonseed oil, sesame oil, olive oil, soybean oil, corn oil, sunflower oil, or peanut oil; a fatty acid esters such as ethyl oleate and isopropyl myristate and variety of other solvent as known per se. The carrier may be chosen based on the physical and chemical properties of the active agent.

In case the active ingredient has a poor water solubility, and an oily carrier is therefore used, proper fluidity can be maintained, for example, by the use of a emulsifiers such as phospholipids, e.g. lecithin or one of a variety of other pharmaceutically acceptable emulsifiers. As known per se, the proper choice if a surfactant and the treatment conditions may also permit to control the particle size of the emulsion droplets.

Suitable soaps for use in parenteral formulations, in case the active ingredient has a poor water solubility, include fatty alkali metal, ammonium, and triethanolamine salts, and suitable detergents include (a) cationic detergents such as, for example, dimethyl dialkyl ammonium halides, and allyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-allyl-imidazoline quaternary ammonium salts, and (3) mixtures thereof.

Further, in order to minimize or eliminate irritation at the site of injection, the compositions may contain one or more nonionic surfactants having a hydrophile-lipophile balance (HLB) of from about 12 to about 17. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

According to yet another aspect, the present invention concerns the use of an active agent selected from the group consisting of an adenosine A1 receptor ligand (A1RL), an adenosine A2 receptor ligand (A2RL), and an adenosine A3 receptor ligand (A3RL) and any combination of A1RL, A2RL and A3RL for modulating GSK-3β activity in cells.

The active agents may be used in the preparation of pharmaceutical compositions for achieving a therapeutic effect in a subject in need, the therapeutic effect comprising modulating GSK-3β activity in cells, the composition comprising a therapeutically effective amount of one or more active agents and a pharmaceutically acceptable additive, the active agent is selected from the group consisting of an adenosine A1 receptor ligand (A1RL), an A2 adenosine receptor ligand (A2RL), an adenosine A3 receptor ligand and any combination of A1RL, is A2RL and A3RL.

As described above, the therapeutic effect may include elevation or suppression of GSK-3β activity. For elevating GSK-3β activity, the active agent is selected from the group consisting of A1RAg, A3RAg, A2RAn and any combination of the same, while for suppressing GSK-3β activity, the active agent is selected from the group consisting of A1RAn, A3RAn, A2RAg and any combination of the same.

Obviously, many modifications and variations of the present invention are possible in-light of tie above teaching. Accordingly, it should be understood that any other effect of modulation of GSK-3β activity in cells, by adenosine receptor ligands, which is within the scope of the appended claims forms part of the present invention and that the invention may be practiced otherwise than as specifically described hereinafter.

### SPECIFIC EXAMPLES

### Example 1

Materials

IB-MECA and MRS1523 were purchased from RBI/Sigma (Natick, Mass., USA). For both reagents stock solution of 10 mM was prepared in DMSO and further dilutions in culture medium were performed to reach the desired concentration; RPMI, fetal bovine serum (FBS) and antibiotics for cell cultures, from Beit Haemek, Haifa Israel. Rabbit polyclonal antibodies against GSK-3β, PKAc, PKB/Akt, c-myc, mouse polyclonal β-catenin and goat polyclonal against β-actin were purchased from Santa Cruz Biotechnology Ic., CA, USA; rabbit polyclonal antibodies against Cyclin D1 which cross reacts with Cycklin D2 were purchased from Upstate Biotchnology Lake Placid, N.Y.; antibodies against phosphorylated PKB/Akt at serine 473 and phosphorylated GSK-3β at serine 9 (rabbit polyclonal), from Cell Signaling Technology, Veverly, Mass., USA. To analyze cAMP, a commercial ELISA kit of cAMP EIA system (Assay Designs Inc., Ann Harbor, USA) was used.

Cells (B16-F10 melanoma cells) were maintained in RPMI medium supplemented with 10% PBS, 200 mM glutamine, 100 U/ml penicillin and 100 μg/ml streptomycin. They were transferred to a freshly prepared medium twice weekly. For all studies we used serum starved cells. FBS was omitted from the cultures for 18 hours and the experiment was carried out on monolayers of cells in RPMI medium supplemented with 1% FBS in a 37° C., 5% CO₂ incubator.

Methods

Cell Proliferation Assay

[³H]-thymidine incorporation assay as used to evaluate cell growth. B136-F10 melanom Cella (1.5×10^{4/}ml) were incubated with IB-MECA (0.0 μM-10 μM) in 96-well microtiter plates for 24 hours. To test whether IB-MECA exerted its effect on tumor cells through binding to A3AR, an antagonist to A3AR, MRS-1523, was added to the cell cultures in the presence of IB-MECA. Cultures of B16-F10 melanoma cells tat were incubated in the presence of MRS-1523 only, served as controls. For the last 6 h of incubation, each well was pulsed with 1 μCi [3H]-thymidine. Cells were harvested and the [³H]-thyine uptake was determined in an LKB liquid scintillation counter (LKB, Piscataway, N.J., USA). These experiments were repeated at least 10 times.

Western Blot Analysis

To detect the level of expression of PKA, GSK-3β (total and phosphorylated), PKB/Akt (total and phosphorylated), β-catenin, c-myc and cyclin D1, protein extract from IB-MECA treated or untreated serum-starved B16-F10 melanoma cells were utilized. To confirm the specificity of IB-MECA to A3AR, 1.01 μM MRS-1523 was added to the culture, 30 minutes prior to the agonist administration. Further, to mimic the chain of events occurring upon activation of A2R, cells were incubated with 8Br cAMP.

At the end of the incubation period, cells were rinsed with ice-cold PBS and lysed in ice-cold lysis buffer (TNN buffer, 50 mM tis buffer pH=7.5, 150 mM NaCl, NP 40). Cell debris was removed by centrifugation at 7500×g for 10 min. Supernatant was utilized for Western Blot analysis. Protein concentrations were determined using the Bio-Rad protein assay dye reagent. Equal amounts of the sample (50 μg) were separated by SDS-PAGE, using 12% polyacryamide gels, The resolved protines were then electroblotted onto nitrocellulose membranes (Schleicher Schuell, Keene, N. H., USA). Membranes were blocked with 1% bovine serum albumin and incubated with the desired primary antibody (dilution 1:1000) for 24 h at 40° C. Blots were then washed and incubated with a secondary antibody for 1 h at room temperature. Bands were recorded using BCIP/NBT color development kit (Promega, Madison, Wis., USA). Data presented in the different figures are representative of at least three different experiments.

Immunohistochemical Staining

Immunohistological staining of IB-MECA treated and untreated B16-F10 melanoma cell specimens was performed according to the following protocol: cells were cultured on Poly-L-Lysine coated glass chamber slides, until they reached approximately 90% confluence, then thy were washed with PBS and fixed with cold acetone for three minutes. Immunocytochemistry was performed using a fluorescent system (Immunofluorescene Kit, Vector Laboratories). Slides were rinsed with PBS and blocked in 1% BSA in PBS containing 5% normal horse or goat serum for 2 hours at room temperature. Then cells were incubated with a primary antibody overnight at room temperature in a humidified chamber. After rinsing with PBS, secondary FITC conjugated antibodies were incubated at room temperature for 1 hr in the dark. Finally, cells were red in PBS, chambers were removed and slides were coverslipped with an aqueous mounting media. Pictures were taken with an ultraviolet microscope using a FITC cube and with a phase filter.

Ribonuclease Protection Assay (RPA)

RPA was performed in order to examine the level of expression of cyclin D1. In this assay RNA was extracted from IB-MECA treated and untreated B-16 melanoma cells. The assay was performed according to instructions by supplier (Pharminogen, San Diago, Calif.) which enabled the generation of a series of templates each of distinct length and representing a sequence in a distinct mRNA species. The probe set was hybridized in excess to target RNA in solution, after which free probe and other single-stranded RNA were digested with RNAases, The remaining “Rnases-protected” probes were purified, resolved by denaturing polyacrylamide gels and quantified by phosphor-imaging. The quantity of each mRNA in the original RNA sample was then determined based on the intensity of the appropriately-sized, protected probe fragment.

Statistical Analysis

The results were statistically evaluated using the Student's t-test. For statistical analysis, comparison between the mean value of different experiments was carried out. The criteria for statistical significance was p<0.05.

Results

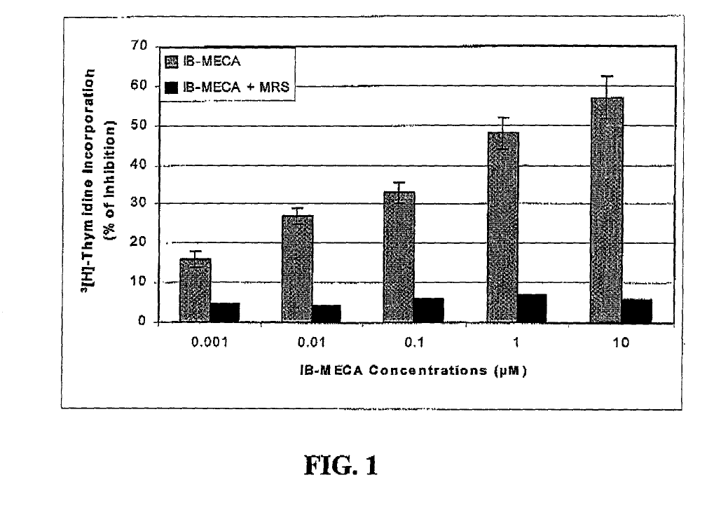

The effect of the A3AR agonist, IB-MECA, on the proliferation of the B 16-F10 melanoma cell line was examined. IB-MECA exerted a dose-dependent inhibitory effect on the growth of melanoma cells. The inhibition of cell growth was statisically significant at all concentrations tested p<0.001). The A3AR antagonist MR-1523 reversed the inhibitory effect of IB-MECA, demonstrating that tumor growth suppression was specifically mediated through A3R (FIG. 1)

The above results led to the determination the signaling pathway involved is downstream to the activation of A3AR. The interaction between the ligand and receptor was evaluated by measuring the production of cAMP, known to be decreased following A3AR activation. Furthermore, PKAc, the downstream element to cAMP, was evaluated. A marked decrease in cAMP level was observed (IB-MECA=5±0.041 pg/ml vs. control=4.2±0.31 pg/ml, p<0.0001), confirming the inhibition of adenylate cyclase activity

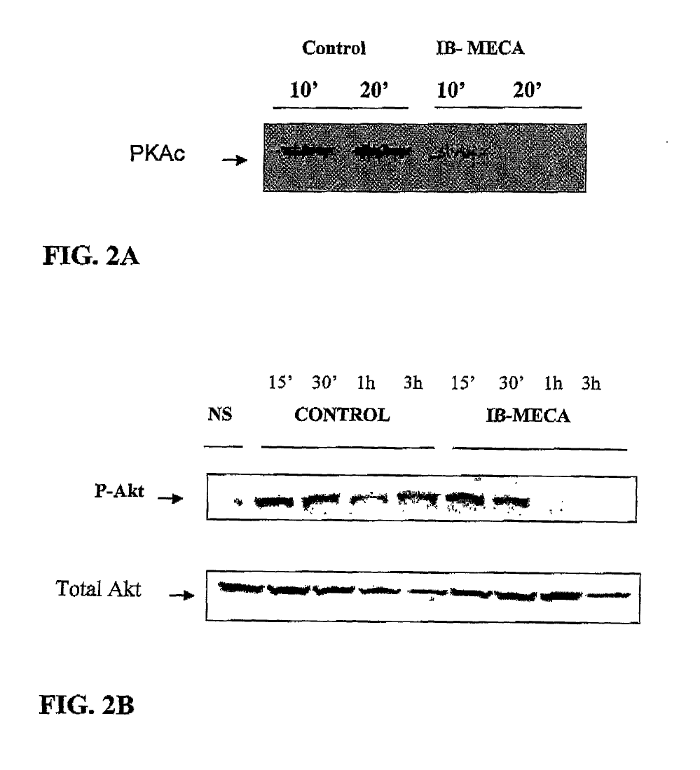

Western blot analysis revealed a decreased level of expression of PKAc upon incubation with IB-NECA for 10 min, followed by a total disappearance of the PKAc band a 20 min (FIG. 2A). Similarly, a decreased level of phosphorylated PKB/Akt was noted after the decrease in the PKAc level (FIG. 2B).

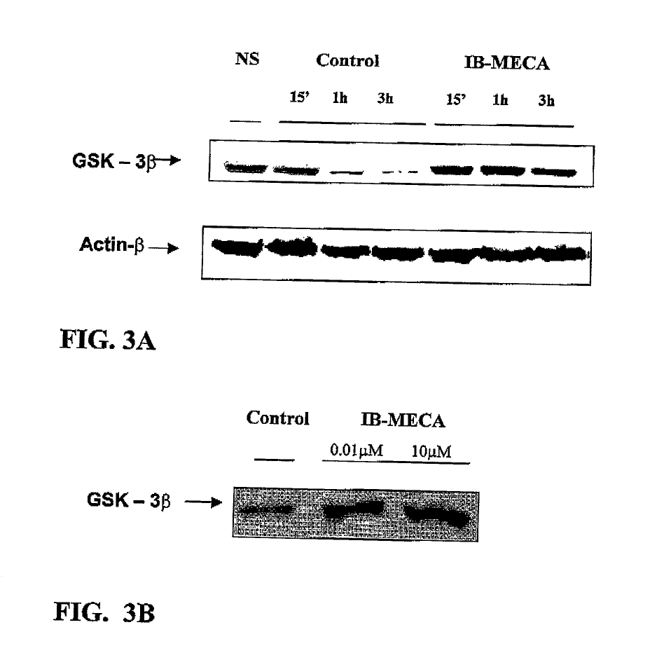

An increase in the total GSK-3β level, at all time points is shown in FIG. 3A, while FIG. 3B shows a dose dependent increase in the level of GSK-3β following treatment of the melanoma cells with 0.01 μM and 10 μM IB-MECA. At the same time, a decrease in the level of phosphorylated GSK-3β (GSK-3β-P) was noted, confirming the finding that active GSK-3β is upregulated (FIG. 3C). The specificity of these responses was concluded when the constant level of the. non-related protein β-Actin, was observed (FIG. 3A). Supportive of these results was the enhanced staining for GSK-3β (immunohistochemistry staining) noted in the IB-MECA treated melanoma cells (results not shown).

Activation of the A3AR inhibits the activity of adenylyl cyclase, leading to decreased cAMP levels. It is suggested that activation of A2AR will result in increased activity of adenylyl cyclase followed by elevation in cAMP levels. In order to evaluate the effect of A2AR activation on the level of GSK-3β downstream events occuring after the receptor activation were mimicked by adding to a culture of B16-F10 melanoma cell 8Br cAMP. A decrease in the level of GSK-3S was noted after the treatment with 8Br cAMP FIG. 3D), suggesting that the expression of GSK-3β can be inhibited by an A2RAg. Therefore, using agonists to the various receptors could alter the levels of GSK-3β leading to opposite responses in the cells.

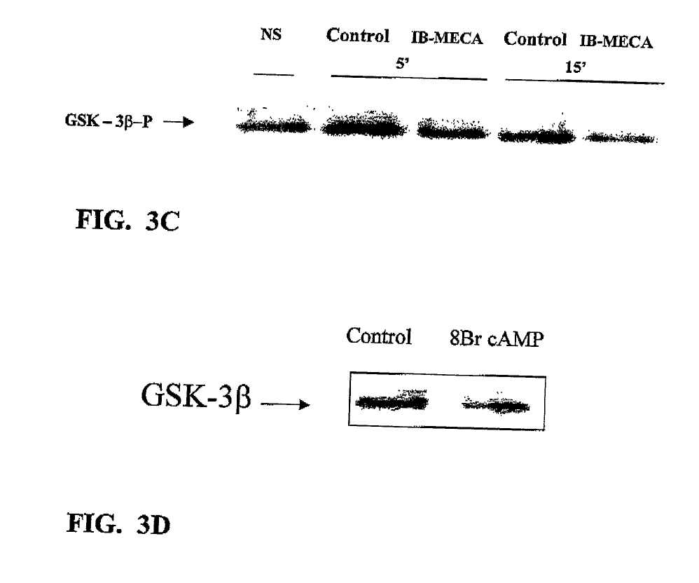

In correlation with the results presented in association with FIG. 1, it has also been shown by an immunoblot assay that the A3RAn, MRS-1523, reversed the decrease in PKAc and the increase in GSK-3β levels FIG. 4), confirming that modulation of kinase activity is mediated via adenosine receptors, such as the A3AR.

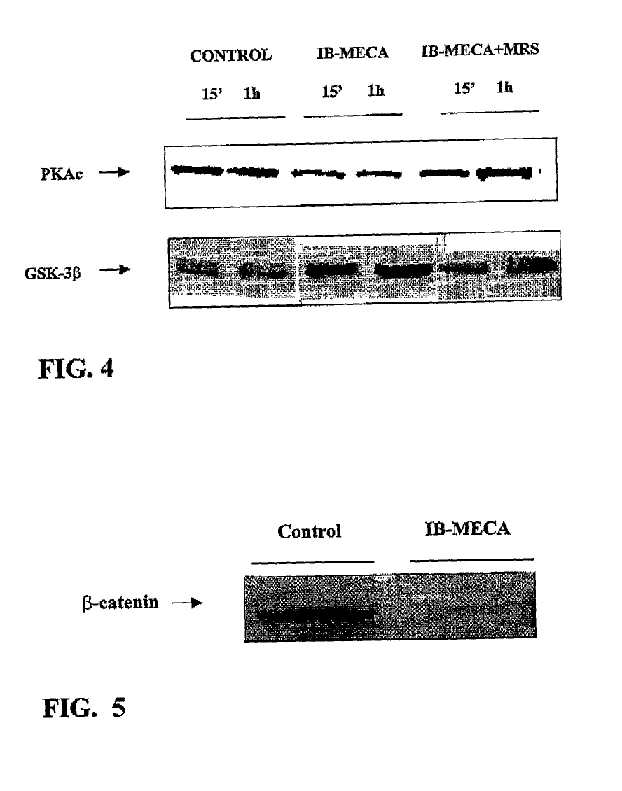

In the light of these results, the level of β-catenin, known to be degraded following phosphorylation by GSK-3β was tested. Indeed a decreased p-catenin level was revealed by Western blot analysis, following treatment of B16-F10 melanoma cells with IB-MECA (FIG. 5). Similar data was exhibited by immunocytological staining, demonstrating a high level of cytoplasmic and nuclear staining of β-catenin in control cells in comparison to decreased staining in IB-MECA treated cells (results not shown).

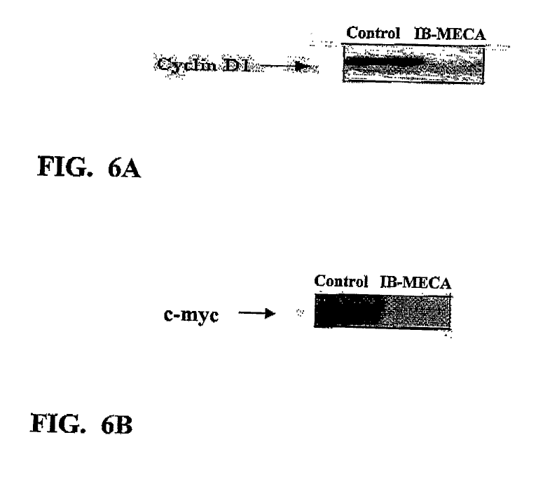

Cyclin D1 and c-myc, known to be transcripted following translocation of β-catin to the cell nucleus, were both found to be down-regulated in tie IB-MECA treated Bb 16-F10 cells (FIGS. 6A and 6B, respectively). Using RPA the cyclin D1 and D2 levels were shown to be decreased in the IB-MECA treated samples (results not shown).

### Example 2

A similar series of studies was utilized in a colon carcinoma murine animal model to determine whether elements of the Wnt pathway altered by IB-MECA in vitro, occur as well in viro with Cl-B-MECA (another A3RAg). The animal model was generated by subcutaneous injection of 1.2×10⁶HCT-116 human colon carcinoma cells to the flank of Balb/C nude mice. The mice were treated orally (by gavage), every second day with 6 μg/Kg Cl-IB-MECA.

After 30 days the mice were sacrificed and tissue samples from the colon carcinoma foci were harvested and analyzed for the expression of β-catenin and cyclin D1.

Results

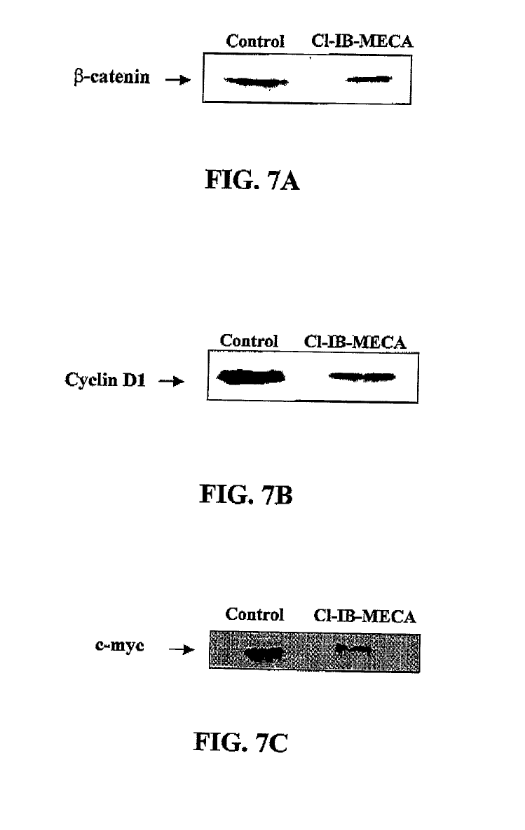

FIGS. 7A, 7B and 7C show that Western immunoblots of protein extracts from tumor tissue, derived from Cl-IB-MECA treated and untreated mice. The results show a decrease in the level of β-catenin, cyc D1 and c-myc, respectively, which is in agreement with in vitro results.

The above described results provide evidence for the participation of the Wnt signaling pathway in A3RAg mediated melanoma and colon carcinoma cell growth in vitro.

It may thus be concluded that A3RAg induces the following events; activation of GSK-3β with a subsequent phosphorylation of β-catenin, leading to its degradation and thereby preventing the migration of β-catenin to the nucleus and the induction of cyclin D1 expression, which eventually leads to cell cycle arrest.

### Example 3

The effect of Cl-IB-MFCA on hair growth was determined, suggesting a connection between hair growth and the Wnt pathway.

Materials

Tumor Cells

Colon carcinoma cells (HCT-116) were employed and were purchased from AITC Rockville, Md. The cells were routinely maintained in RPMI medium containing 10% fetal bovine serum (PBS, Biological Industries, Beit Haemek, Israel. Twice a week the cells were transferred to a freshly prepared medium.

Nude Mice

Nude mice (BalbC origin) were subcutaneously inoculated with HCT16 human colon carcinoma cells, which thus developed a visible tumor.

Drugs

Cl-IB-MECA was dissolved in DMSO and kept as a stock solution. in a concentration of 10 mM. Before administration to the mice, the stock solution was diluted with PBS to a concentration so that each mice received a dosage of 6 μg/kg body weight.

Methods

Nude mice (BalbC origin) were subcutaneously inoculated wit HCT-16 human colon carcinoma cells. These mice were divided into two groups:

Study group which was daily and orally administered with Cl-IB-MECA (6 μg/kg body weight) dissolved in DMSO and diluted with PBS.

Control group which was daily and orally administered with DMSO only.

Results

After 30 days of daily treatment, hair grew on the skin of the nice from the study group. These results suggest that adenosine agonists, such as Cl-IB-MECA employed in tis particular, non-limiting example, are potential agents in treating or preventing hair loss as well as agents for inducing hair growth, through the activation of GSK-3β in the Wnt pathway.

### Example 4

Materials and Methods

Preparation of Primary Human Fetal Astrocytes and Microglia

Purified primary human fetal astrocytes and microgial cells were prepared from 16 to 20 week old human fetal brain tissue by a modified procedure based on the methods of Cole and de Vellis [R. Cole and J. de Vellis. In: Protocols for neural cell cure. S. Fedoroff and A. Richardson (Eds.) Human Press, Totowa, N.J., pp. is 117-130. (1997)], and Yong and Antel [V. W. Yong and J. P. Antel. In: Protocols for neural cell culture. S. Fedoroff and A. Richardson (Eds.) Humana Press, Totowa, N.J., pp. 157-172 (1997)]. Brain tissue was washed in ice-cold Hank's Balanced Salt Solution (HBSS) containing the antibiotics gentamycin and arnphotericin B. Blood vessels and meninges were removed and the tissue was minced into small pieces. After mincing, the tissue was enzymatically dissociated by incubation in 0.05% trypsin and mechanically disrupted by passing several times over a 75 μm nylon mesh filter. The resulting single cell suspension was washed, pelleted and plated at a density of 2-10×10⁶ cells per 162 cm² flask in DMEM:F12 containing 10% fetal calf insulin, gentamycin, and L-glutamine. After 7-10 days of growth, microglial cells were isolated by placement on rotary shaker at 200 rpm in a 37° C. incubator overnight. The non-adherent cells were removed and allowed to attach to a new flask for 1 to 3 h. Following attachment, the cells were washed and refed with media containing 10% fetal calf serum, insulin, gentamycin, L-gluntamine, and N1 supplement. Astocyes were subeultured from adherent cells in media containing 15% fetal calf serum, insulin, gentamycin, and L-glutamine and contaminating microglia were removed by repeated rotary shaking. Cultured astrocytic and nicroglial cells were plated at a density of 2.5×105 per well into 6 well plates for subseqent infection.

Preparation of HIV-1 Virus

Brain derived primary HIV-1 isolates SF162 and JR-FL were cultured in human peripheral blood mononuclear cells (PBMC) essentially as described by Gartner and Popovic¹². PBMC were isolated from human buffy coat by ficoll gradient and plated at a density of 2.5×10⁶ per ml in RPMI containing 10% fetal calf serum and gentamycin. Cells were stimulated by the addition of 5 μg/ml of phytohemagglutinin (PHA) for 48 h. After stimulation, cells were infected with either SF162 or JR-FL and cultured for 7 to 10 days until high titres of MIV-1 were detected in the supernatant by p24 ELISA assay. When viral production was optimal, the cells were pelleted, the supernatant containing HIV-1 was aliquoted and stored at −70° C. until use. P24 ELISA assay was performed on an aliquot of stock to determine the viral titre.

Infection of Primary Human Fetal Astrocytic and Microglial Cells and Treatment with Cl-IB-MECA.

Microglial or astrocytic cells (2.5×10⁵) were plated per well into 6-well plates. The next day, cells were washed and refed with fresh medium. 2'10⁴ p24 units of either SF 162 or JR-FL virus was added per well in a total of 1 ml of viral inoculum. In control experiments, the virus was not added. Cells were incubated with virus overnight at 3720 C., washed extensively with PBS, and re-fed with 2 ml fresh medium. Cultures were treated with IB-MECA or Cl-IB-MECA at a concentration of 0.01 μM every 24 hours, 500 μl of medium were removed at the indicated times following infection and stored at -70° C. Nor later analysis. Each time medium was removed, a volume amount of fresh medium was added. In control experiments IB-MECA and Cl-IB-MECA were omitted.

p24 ELISA Assay

ELISA assay to detect the HIV-1 viral core protein, p24, was performed on 50 μl of the collected supernatant utilizing the commercially available p24 ELISA Kit (NEN/Dupont) according, to the manufacturer's instructions.

Results

A seen in Tables 1 to 3, the amount of p24 protein present in culture medium collected from HIV infected cells is significantly reduced in HIV infected cells treated with IB-MECA (HIV and IB-MECA) or Cl-IB-MECA (HIV and Cl-IB-MECA) in comparison to controls not treated with either IB-MECA or Cl-IB-MECA. (HIV).

Table 1 shows the effect of IB-MECA and Cl-IB-MECA on HIV replication n JR-FL infected astroglial cells, wherein p 24 protein (pg/mL) was measured in medium from cell cultures 5 days after HIV infection.

Table 2 shows the effect of IB-MECA and Cl-IB-MECA on HIV replication in SF162 infected astroglia, wherein p 24 protein (pg/mL) was measured as indicated above.

Table 3 shows the effect of IB-MECA and Cl-IB-MECA on HIV replication in SF126 infected microglia/SF, wherein p 24 protein (pg/mL) was measured in medium from cell cultures 5 days and 10 days after HIV infection.

TABLE 1
Astroglia/JR-FL

| Treatment | p 24 pg/mL Day 5 |
|---|---|
| No HIV | 12.64 |
| HIV | 22.83 |
| HIV and IB-MECA 0.01 | 3.02 |
| HIV and Cl-IB-MECA 0.01 | 8.45 |

TABLE 2
Astroglia/SF-162

| Treatment | p 24 pg/mL Day 5 |
|---|---|
| No HIV | −12.96 |
| HIV | 313.38 |
| IB-MECA 0.01 | 137.58 |
| Cl-IB-MECA 0.01 | 288.77 |

TABLE 3
Microglia/SF

| | p 24 pg/mL | |
|---|---|---|
| | Day 5 after infection | Day 10 after infection |
| No HIV | −12.64 | −12.64 |
| HIV | 267.99 | 209.18 |
| IB-MECA 0.01 | 81.33 | 62.79 |
| IB-MECA 0.1 | 82.29 | 54.80 |
| Cl-IB-MECA 0.01 | 127.03 | 111.05 |
| Cl-IB-MECA 0.0.1 | 10.81 | 80.37 |

These results suggest that Cl-IB-MECA and IB-MECA, well known A3RAg, inhibit viral replication. It is further suggested that this inhibitory effect is involved in GSK-3β modulation/

Discussion

The inhibition of tumor cell growth by adenosine and its A3AR agonists, IB-MECA and Cl-IB-MECA was already described⁽⁷⁾.

It has now been shown that activation of A3AR plays a role in signal transduction pathways.

IB-MECA inhibited the proliferation of B16-F10 melanoma cells in a dose dependent manner. Administration of the A3AR antagonist MRS-1523, to the culture system, reversed most of the inhibitory effect, indicating that IB-MECA's activity was mediated through A3AR.

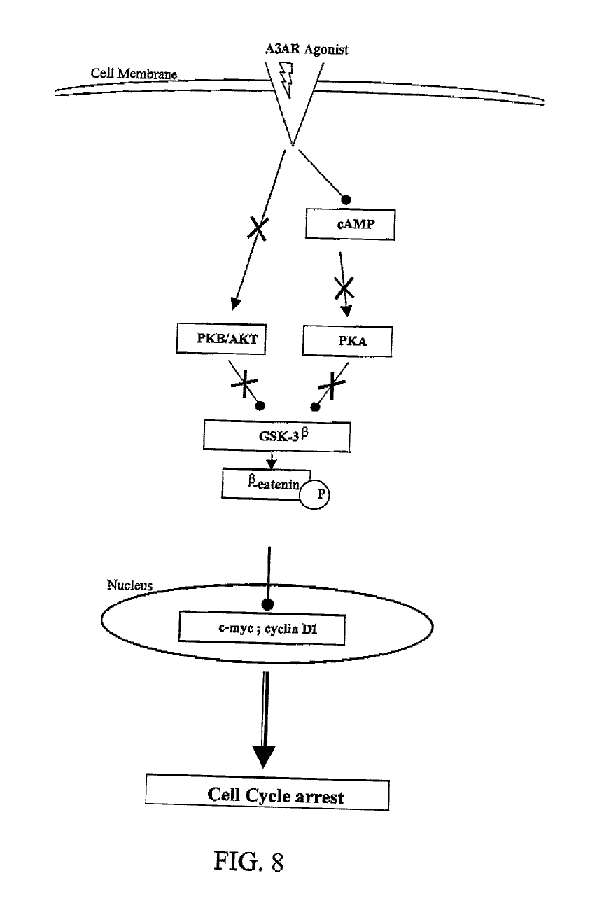

One of the signal transduction pathways controlling cell cycle progression is Wnt, which is highly active during embryogenesis and tumorigenesis. In a number of neoplasia, including malignant melanoma, GSK-3β fails to phosphorylate β-catenin. The stabilized β-catenin accumulates in the cells, locates to the nucleus where it binds to Lef/Tcf family of transcription factors and up-regulates the expression of WNt target genes including cyclin D1 and c-myc. GSK-3β thus has a prominent role in the pathway since it. modulates the level of β-catenin. FIG. 8 provides a schematic illusion. of the Wnt pathway activation by A3RAg.

Two effector proteins, PKB/Akt and PKA, control the level and activity of GSK-3β and therefore, indirectly, are involved in the regulation of the Wnt pathway. Both arc capable of phosphorylating GSK-3β at serine 9 and 21, inducing its inactivation and inability to phosphorylate β-caterin. It was established that cAMP activates PKA by dissociating the, PKAc unit from the parent molecule. PKB/Akt is known to be activated in response to stimulation with various growth factors through a phosphatidylinositol 3′-kinase (P13-kinase) dependent pathway⁽⁸⁾. However, Fillipa et al.⁽³⁾ have shown tat PKB/Akt is also activated by cAMP elevating agents. This group demonstrated that PKAc (following activation by cAMP), is able to phosphorylate and activate PKB/At, in 293 human kidney embryonic cells, in a pathway independent of P13K.

It has now been shown and disclosed that activation on of A3AR by IB-MECA or by Cl-IB-MECA induced a reduction in the formation of camp, which subsequently decreased PKAc and PKB/Akt levels and that this activity may be blocked by a A3RAn or by an A2RAg. It is therefore sugested that PKAc and PKB/Akt downregulation suspends GSK-3β phosphorylation thereby evening GSK-3β to its active form. Supporting this notion, was the decrease in the level of phosphorylated GSK-3β that was found following IB-MECA or Cl-IR-MECA treatment.

Specificity of the cAMP/GSK-3β pathway was confirmed by introduction of MRS-1523 to the IB-MECA-treated B16-F10 cultures, which prevented changes in the levels of PKAc and GSK-3β.

Following these events a reduction in β-catenin levels, with a subsequent decreased c-myc and cyclin D1 levels, was observed. Cell cycle progression is controlled by cyclin dependent kinases, whose activities are regulated by a series of cyclins. Cyclin D1 and cyclin D2 have been reported to peak in the early and late, respectively, G1 phase. Results showing decreased mRNA and protein levels of cyclin D1 and D2 upon IB-NECA treatment, suggest their important role in the G1/S transition.

GSK-3p was shown to directly phosphorylate cyclin D1 on Thr-286, thereby triggering rapid cyclin D1 turnover [Diehl JA, et al. Genes Dev 12:3499-3511 (1998)]. The stimulatory effect of IB-MECA on GSK-3β activity may induce the decrease in cyclin D1 level through that pathway.

Taken together, adenosine receptors, such as A3RAg (e.g. IB-MECA or Cl-IB-MECA) orchestrate a chain of events starting at the receptor level where it downregulates cAMP, PKAc and PKB/Akt, thus enabling the activation of GSK-3β, the key element of Wnt. The ability of adenosine receptor ligands to interfere with the Wnt pathway suggests they may be applied to cancer therapy as well as to other disorders which require for their treatment modulation of the GSK-3β activity. For example, the involvement of GSK-3β in the mechanism of a variety of other clinical situations has been described. It is responsible for tau phosphorylation in neuronal cells which are implicated in the etiology of Alzheimer's disease, it is overexpressed in Diabetes type II and in HIV infected cells [Aggirwar SB, et at. J. Nemrochem 73:578-86, (1999); Niloulina S. E., et al. Diabetes 49,263-271 (2000)]. The modulation of cellular GSK-3β levels through the activation (agonist) or blockade (antagonist) of the receptor, enables the use of this receptor as a target to combat disease mechanisms which arise from or involve up or downregulation of GSK-3β.

1. A method for a therapeutic treatment, comprising administering to a subject in need an effective amount of an active agent for achieving a therapeutic effect, the therapeutic effect comprises modulating GSK-3β activity in cells and said active agent is an adenosine receptor ligand (ARL).

2. The method of claim 1, wherein said modulation involves activation of GSK-3β activity and said ARL is selected from an adenosine A1 receptor agonist (A1RAg), an adenosine A3 receptor agonist (A3RAg), an adenosine A2 receptor antagonist (A2RAn) or a combination of the same.

3. The method of claim 1, wherein said modulation involves inhibition of GSK-3β activity and said ARL is selected from an adenosine A1 receptor antagonist (A1RAn), an adenosine A3 receptor antagonist (A3RAn), an adenosine A2 receptor agonist (A2RAg) or a combination of the same.

4. The method of claim 2, wherein said ARL is A1RAg.

5. The method of claim 4, wherein said A1RAg is selected from the group consisting of N⁶-cyclopentyl adenosine (CPA), 2chloro-CPA (CCPA), N⁶-cyclohexyl adenosine (CHA), N6-(phenyl-2R-isopropyl)adenosine (R-PIA) and 8-{4[({[(2-aminoethyl)amino]carbonyl}methyl)oxyl-phenyl}-1,3-dipropylxanthine (XAC).

6. The method of claim 2, wherein said ARL is an adenosine A1 receptor agonist (A3RAg).

7. The method of claim 6, wherein said A3RAg is selected from the group consisting group consisting of 2-(4-aminophenyl)ethyladenosine (APNEA), N⁶-(4-amino-3-iodobenzyl)adenosine-5′-(N-methylmronamide) (AB-MECA), N⁶-(2-iodobenzyl)-adenosine-5′-N-methly-uronamide (IB-MECA) and 2-chloro-N⁶-(2-iodobeonyl)-adenosine-5′-N-methly-uronamide (Cl-IB-MECA).

8. The method of claim 6, wherein said A3RAg is Cl-IB-MECA.

9. The method of claim 6, wherein said ARL is a xanthine-7-riboside derivative.

10. The method of claim 2, wherein said ARL is an adenosine A2 receptor antagonist (A2RAn).

11. The method of claim 10, wherein said A2RAn is 3,7-dimethyl-1-propargyl-xantane (DMPX).

12. The method of claim 2, for the treatment of a disease or disorder which requires for its treatment elevation of GSK-3β activity.

13. The method of claim 12, wherein said disorder is hair loss.

14. The method of claim 3, wherein said ARL is an A1RAn.

15. The method of claim 14, wherein said A1RAn is 1,3-dipropyl-8-cyclopentylxantine (DPCPX).

16. The method of claim 3, wherein said ARL is an A3RAn.

17. The method of claim 16, wherein said A3RAn is selected from the group consisting of 5-propyl-2-ethyl4-propyl-3-ethylsulfinylcarbonyl)-6-phenylpyridine-5-carboxylate (MRS-1523) and 9-chloro-2-(2-furanyl)-5-[(phenylacetyl)amino][1,2,4,]-triazolo[1,5-c]quinazoline (MRS-1200).

18. The method of claim 3, wherein said ARL is an adenosine A2RAg.

19. The method of claim 18, wherein said AM2Ag is N⁶-[2-(3,5-dimethoxyphenyl)-2-(2-methylphenyl)-ethyl]adeosine (DMPA).

20. The method of claim 3, for the treatment of a disease or disorder which requires for its treatment suppression of GSK-3β activity.

21. The method of claim 20, wherein said disease is a disease associated with degeneration of cells.

22. The method of claim 20, wherein said disease is a neurodegenerative disease or a neurotraumatic disorder.

23. The method of claim 20, wherein said disorder is associated with psychiatric disorders.

24. The method of claim 20, wherein said disease is non-insulin dependent diabetes mellitus.

25. The method of claim 1, wherein said active agent is administered orally.

26. A pharmaceutical composition for achieving a therapeutic effect in a subject in need, the therapeutic effect comprising modulating GSK-3β activity in target cells, the composition comprising a therapeutically effective amount of at least one active agent and one or more pharmaceutically acceptable additives, said active agent is an adenosine receptor ligand (ARL).

27. The composition of claim 26, wherein said modulation involves activation of GSK-3β activity and said ARL is selected from an adenosine A1 receptor agonist (A1RAg), an adenosine A3 receptor agonist (A3RAg), an adenosine A2 receptor antagonist (A2RAn) or a combination of the same.

28. The composition of claim 26, wherein said modulation is inhibition of GSK-3β activity and said ARL is selected from an adenosine A1 receptor antagonist (A1RAg), an adenosine A3 receptor antagonist (A3RAg), an adenosine A2 receptor agonist (A2RAg) or a combination of the same.

29. The composition of claim 27, wherein said ARL is an A1RAg.

30. The composition of claim 29, wherein said A1RAg is selected from the group consisting of the N⁶-cyclopentyl adenosine (CPA), 2-chloro-CPA (CCPA), N⁶-cyclohexyl adenosine (CHA), N6-(phenyl-2R-isopropyl)adenosine (R-PIA) and 8-{4-[({[(2-aminoethyl)amino]carbonyl}methyl)oxyl-phenyl}-1,3-dipropylxanthine (XAC).

31. The composition of claim 27, wherein said ARL is an A3RAg.

32. The composition of claim 31, wherein said A3RAg is selected from the group consisting group consisting of 2-(4-aminophenyl)ethyladenosine (APNEA), N⁶-(4-amino-3-iodobenzyl) adenosine-5′-(N-methyluronade) (AB-MECA), N⁶-(2-iodobenzyl)-adenosine-5′-N-methly-uronamide (IB-MECA) and 2-chloro-N⁶-(2-iodobenzyl)-adenosine-5′-N-methyl-uronamide (Cl-IB-MECA).

33. The composition of claim 32, wherein the A3RAg is Cl-IB-MECA.

34. The composition of claim 31, wherein ARL is a xanthine-7-riboside derivative.

35. The composition of claim 27, wherein said ARL is an A2RAn.

36. The composition of claim 35, wherein said A2RAn is 3,7-dimethyl-1-propargyl-xantane (DMPX).

37. The composition of claim 27, for the treatment of a disease or disorder which requires for its treatment elevation of GSK-3β activity.

38. The composition of claim 37, for the treatment of hair loss.

39. The composition of claim 28, wherein said active agent is an 3RAn.

40. The composition of claim 39, wherein said A3RAn is 5-propyl-2-ethyl-4-propyl-3-ethylsuifanylcarbonyl)-6-phenylpyridine-5-carboxylate (MRS-1523) and 9-chloro-2-(2-furanyl)-5-[(phenylacetyl)amino][1,2,4,]-triazolo[1,5-c]quinazoline (MRS-1200).

41. The composition of claim 28, wherein said ARL is an A1RAn.

42. The composition of claim 41, wherein said A1RAn is 1,3-dipropyl-8-cyclopetyxanthine (DPCPX).

43. The composition of claim 28, wherein said ARL is an A2RAg.

44. The composition of claim 43, wherein said A2RAg is N⁶-[2-(3,5-dimethoxyphenyl)-2-(2-methylphenyl)-ethyl]adenosine (DMPA).

45. The composition of claim 26, for the treatment of a disease or disorder which requires for its treatment suppression of GSK-3β activity.

46. The composition of claim 45, wherein said disease is a disease associated with degeneration of cells.

47. The composition of claim 45, wherein said disease is a neurodegenerative disease or a neurotraumatic disorder.

48. The composition of claim 45, wherein said disorder is associated with psychiatric disorders.

49. The composition of claim 45, wherein said disease is non-insulin dependent diabetes mellitus.

50. The composition of claim 26, formulated for oral administration.

51. Use of an adenosine receptor ligand (ARL) for modulating GSK-3β activity in cells.

52. Use according to claim 51, for elevating GSK-3β activity, wherein said ARL is selected from A1RAg, A3RAg, A2RAn or any combination of the same.

53. Use according to claim 51, for suppressing GSK-3 activity, wherein said ARL is selected from A1RAn, A3RAn, A2RAg or any combination of the same.

54. Use of an adenosine receptor ligand (ARL) for the preparation of a pharmaceutical composition for tie treatment of a disease or disorder which requires for its treatment suppression of GSK-3β activity.

55. Use according to claim 51, substantially as described in the specification.
